(19) United States
(12) Patent Application Publication
(10) Pub. No.: US 2016/0324565 A1
(43) Pub. Date: Nov. 10, 2016

(54) Cervical Seal

(71) Applicant: BOSTON SCIENTIFIC SCIMED, INC., Maple Grove, MN (US)

(72) Inventors: Patrick NICOLAS, Needham, MA (US); Doreen RAO, Sudbury, MA (US); Michael S.H. CHU, Brookline, MA (US); John PETRICCA, Ashland, MA (US)

(21) Appl. No.: 15/216,504

(22) Filed: Jul. 21, 2016

Related U.S. Application Data
(63) Continuation of application No. 12/729,802, filed on Mar. 23, 2010, now abandoned.
(60) Provisional application No. 61/166,035, filed on Apr. 2, 2009.

(51) Int. Cl. A61B 18/04

(52) U.S. Cl. CPC A61B 18/04; A61B 2018/046; A61B 2018/00577

(57) ABSTRACT

A thermal ablation device includes an elongated member sized and shaped for insertion into a uterus via a cervical opening. The elongated member defines a first lumen extending therethrough to a first distal opening which, when the apparatus is in an operative position, opens into the uterus and a seal positioned on the elongated member so that, when the elongated member is in the operative position, the seal engages cervical tissue around a circumference of the elongated member and prevent fluids introduced into the uterus via the first lumen from flowing therepast into the cervix. The seal includes a plurality of flexible members separated from one another along a length of the elongated member and projecting radially outward from an outer surface of the elongated member.

### PRIORITY CLAIM

This application claims the priority to the U.S. Provisional Application Ser. No. 61/166,035, entitled “Cervical Seal” filed on Apr. 2, 2009. The specification of the above-identified application is incorporated herewith by reference.

### BACKGROUND

or dysfunctional uterine bleeding may arise from a hormonal imbalances, systemic disease or anatomical abnormality such as fibroids, polyps or other growths. A common non-invasive method means of treating such conditions comprises ablation of the lining of the uterus. In hydro-thermal ablation (“HTA”), heated saline solution is applied to targeted portions of the endometrial lining via a hysteroscope inserted into the uterus through the cervical canal. However, leakage of the heated fluid from the uterus can damage non-targeted tissue.

### SUMMARY OF THE INVENTION

The present invention is directed to a thermal ablation device, comprising an elongated member sized and shaped for insertion into a uterus via a cervical opening, the elongated member defining a first lumen extending therethrough to a first distal opening which, when the apparatus is in an operative position, opens into the uterus and a seal positioned on the elongated member so that, when the elongated member is in the operative position, the seal engages cervical tissue around a circumference of the elongated member and prevent fluids introduced into the uterus via the first lumen from flowing therepast into the cervix, the seal including a plurality of flexible members separated from one another along a length of the elongated member and projecting radially outward from an outer surface of the elongated member.

### BRIEF DESCRIPTION OF DRAWINGS

FIG. 1 shows a known ablation device;

FIG. 2 shows a perspective view of an ablation system according to an embodiment of the invention;

FIG. 3 shows a perspective view of a distal end of the system of FIG. 2;

FIG. 4 shows a side view of the cervical seal of the distal end of the system of FIG. 2;

FIG. 5 shows a side view of the distal end of FIG. 2;

FIG. 6 shows a perspective view of the cervical seal of FIG. 4;

FIG. 7 shows a partially cross-sectional view of a distal end of an ablation system according to a further embodiment of the invention;

FIG. 8 shows a partially cross-sectional view of a distal end of an ablation system according to a still further embodiment of the invention;

FIG. 9 shows a perspective view of a cervical seal for use with an ablation system according to a fourth embodiment of the invention;

FIG. 10 shows a cross-section of a distal end of an ablation system including the cervical seal of FIG. 9;

FIG. 11 shows a perspective view of a cervical seal for use with an ablation system according to a fifth exemplary embodiment of the invention;

FIG. 12A shows a perspective view of a cervical seal or use with an ablation system according to a sixth exemplary embodiment of the invention;

FIG. 12B shows a perspective view of a cervical seal for use with an ablation system according to a seventh exemplary embodiment of the invention;

FIG. 12C shows a perspective view of a cervical seal for use with an ablation system according to a eighth exemplary embodiment of the invention;

FIG. 13A shows a first perspective view of a cervical seal for use with an ablation system according to a ninth exemplary embodiment of the invention;

FIG. 13B shows a second perspective view of the cervical seal of FIG. 13A; and

FIG. 14 shows an expansion device for insertion of a cervical seal according to an exemplary embodiment of the present invention.

### DETAILED DESCRIPTION

The present invention may be further understood with reference to the following description and the related appended drawings, wherein like elements are provided with the same reference numerals. The present invention describes a sealing device for preventing the leakage of fluids from the uterus when performing hydrothermal ablation. An exemplary sealing device of the present invention is directed for use with standard HTA trans-cervical sheaths. However, it is noted that the present invention may be employed to enhance the sealing for other ablation device inserted into hollow organs via openings which naturally seal around the devices inserted therethrough.

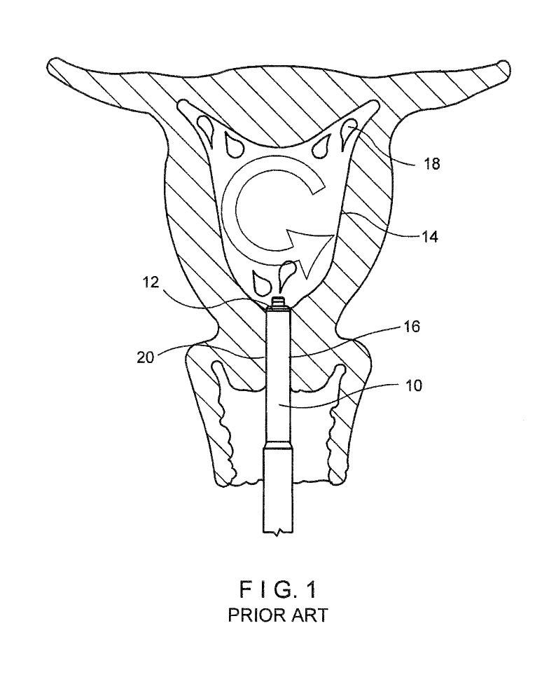

FIG. 1 depicts a known ablation device 10 deployed in an active configuration with a distal tip 12 thereof opening into a uterus 14 of a living body. The ablation device 10 is includes an elongated tubular member 16 with a substantially smooth outer portion sized to fit within the cervical canal 20. A maximum diameter of the ablation device 10 is maintained less than a predetermined maximum diameter to prevent undue trauma to the cervical canal 20. To sealingly engage the cervical walls of most patients without overly stretching the tissue thereof, an outer diameter of the ablation device 10 may, for example, be approximately 7.62 mm. If the outer diameter of the ablation device 10 is insufficient to sealingly engage the walls of the cervical canal 20, the heated fluid 18 may leak out of the uterus 14 and damage non-targeted tissue while decreasing the efficacy of the ablation procedure. A proximal portion of the ablation device 10 further comprises an increased diameter portion which prevents, for example, an insertion of the ablation device 10 into the cervical canal 20 beyond the length of the elongated tubular member 16.

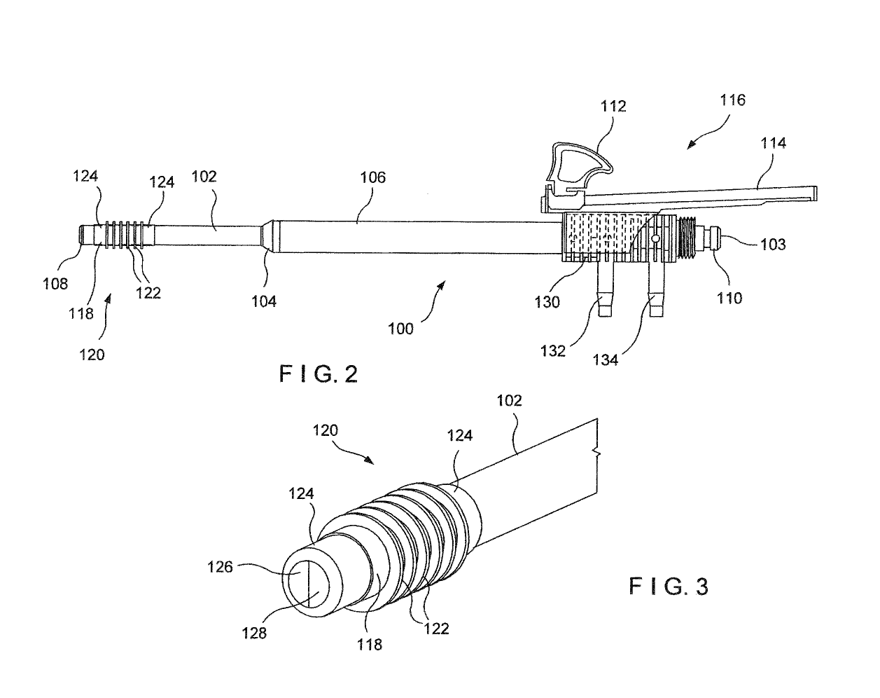

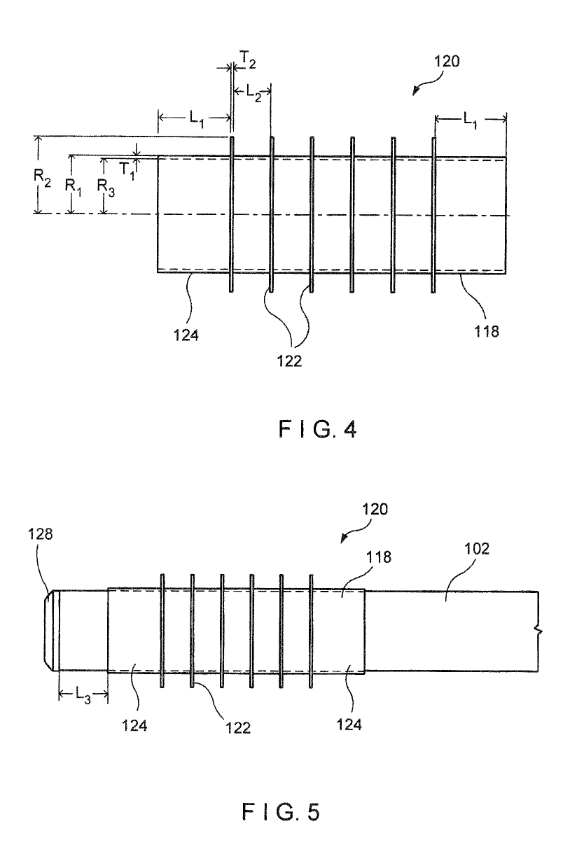

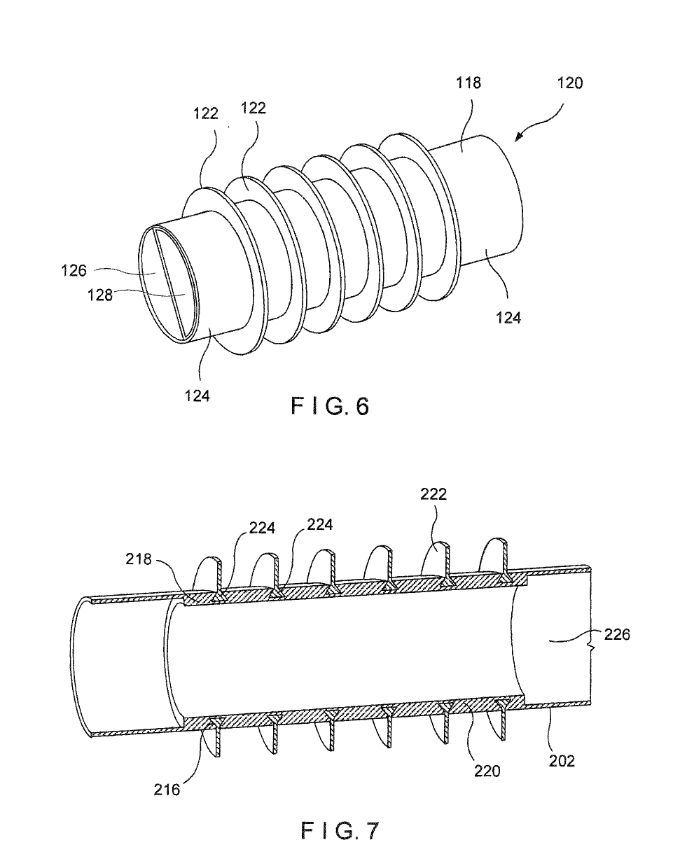

As shown in FIGS. 2-6, an introducer 100 for an ablation system according to an exemplary embodiment of the invention comprises an elongated body 106 extending from a proximal end 103 which remains outside the body at all times to a distal end 108 which, when in an operative position opens into the uterus 14 via the cervical canal 20 in the same manner as the distal tip 12 of the device 10. In use, as would be understood by those skilled in the art, the introducer 100 is coupled to a known thermal ablation system including a supply of heated fluid and a system for withdrawing the fluid from the body. For example, the introducer 100 of the present invention may be employed with a thermal ablation system as taught in U.S. Patent Application Ser. No. 60/987,913, entitled “Thermal Ablation System”, naming as inventors Robert Bouthillier, Michael P. Fusaro, Joseph M. Gordon, Stephen S. Keaney, Brian Maclean, Andrew W. Marsella, David Robson and Boris Shapeton filed Nov. 14, 2007, the entire disclosure of which is hereby incorporated by reference herein. Generally, the thermal ablation system may include a console receiving fluid from an intravenous (IV) bag (not shown) supplying ablation fluid thereto at a desired pressure. The IV bag may contain fluid such as saline that will be heated and circulated through the uterus to ablate the endometrial lining The fluid may be filtered and recirculated or removed from the system after being withdrawn from the body as would be understood by those skilled in the art. During the ablation procedure, an operator (e.g., physician, nurse, etc.) may be required to substitute the IV bag for IV bags with other fluids depending on stage of the ablation procedure. For example, prior to the procedure an anesthetic fluid may be circulated through the uterus to numb the endometrium. Additionally, after the endometrial lining has been ablated, an analgesic and/or infection preventative solution may be circulated through the uterus. The console comprises a housing encasing electronic circuitry and providing a user interface for displaying content (e.g., instructions, procedural data, warnings, etc.) and receiving user input. The user interface may comprise a display screen (e.g., LCD) and a keypad for submitting input to the console. The console effectively controls the flow of fluid into and out of the introducer 100.

As shown in FIG. 2, an introducer 100 according to an exemplary embodiment of the present invention is coupled to a fluid source (not shown) and a fluid reservoir (not shown) via a delivery inlet 134 and a return outlet 132 to deliver fluid to the uterus 14 and to return the fluid from the uterus 14 to the fluid reservoir (not shown). The delivery inlet 134 and the return outlet 132 are respectively coupled to a delivery lumen 126 and a return lumen 128 both extending longitudinally through the introducer 100. The delivery lumen 126 and the return lumen 128 terminate at a distal end 108 of the sheath 102 which, when the introducer 100 is in an operative position, is located within the uterus. The delivery lumen 126 and the return lumen 128 are formed of substantially equal sizes to permit an even circulation of ablation fluid through the uterus. Although the delivery lumen 126 and the return lumen 128 as concentric tubes, it is submitted that lumens of any suitable shape may be employed without deviating from the spirit and scope of the present invention such as, for example, circular lumens, elliptical lumens, etc. The introducer 100 may optionally include a vision system to allow visualization of the operative area. Those of skill in the art will understand that the vision system may be substantially similar to the systems in conventional endoscopes (e.g., fiber optic or CCD-based systems). Alternatively, users may rely on the vision system of an endoscope or other instrument inserted through the introducer 100, as those skilled in the art will understand.

The introducer 100 includes a handle 130 coupled to the sheath 102 and comprising a scope connector 110 for receiving a visualization device, such as an endoscope. The handle 130 facilitates holding and manipulation of the introducer 100 with a single hand while the operator uses his free hand to interface with a console (not shown) controlling the visualization device, manipulate the patient's anatomy, etc. The scope connector 110 according to this embodiment is disposed on a proximal end of the introducer 100 and provides an attachment point for the visualization device (e.g., a hysteroscope, an endoscope) so that the visualization device may be passed distally through a visualization lumen in the introducer 100 and extended out of the distal end 108. Thus, the operator may visually monitor insertion of the introducer 100 into the uterus.

The visualization device is inserted into the proximal end of the introducer 100 through the scope connector 110 and locked thereto using a means known in the art. Those of skill in the art will understand that the scope connector 110 may be implemented as any mechanism which allows the length of the introducer 100 to be adjusted to and maintained at a new length. For example, a scope adapter (not shown) may be provided as a projection on an outer surface of a distal portion of the scope connector 110 received in and movable between one of a number of locking apertures (not shown) formed on the handle 130. Partial barriers may be formed between each of the locking apertures (not shown) to retain the scope adapter (not shown) in a selected one of the locking apertures (not shown) maintaining a selected length of a portion of the scope connector 110 projecting from the proximal end of the introducer 100. In another embodiment, a rack (not shown) may be formed on the distal portion of the scope connector 110 mating with a gear in the handle 130 so that rotation of the gear extends and withdraws the scope connector 110 relative to the handle 130. A ratchet may be provided to maintain the gear in a fixed position relative to the rack, thereby maintaining the desired position of the scope connector 110 relative to the handle 130.

The introducer 100 of the present invention may also comprise a tenaculum stabilizer 116 on the handle 130. During the ablation procedure, a tenaculum (not shown) may be placed around the cervical canal and coupled to the ablation device 100 to ensure that the ablation device 100 remains at a desired position and depth within the uterus and is not inadvertently withdrawn therefrom. That is, the tenaculum stabilizer 116 ensures that the distal end 108 of the sheath 102 is not withdrawn proximally from the uterus during the procedure to prevent non-targeted tissue from being exposed to the ablation fluid. When the tenaculum is coupled to the tenaculum stabilizer 116, the operator selects a tension to be applied between the tenaculum and the ablation device 100 by moving the fin along the rail 114. A positioning mechanism (e.g., ratchet, latch, clip, etc.) may be used to maintain a position of a fin 112 relative to the rail 114, as would be understood by those skilled in the art. Such a tenaculum stabilizer device is also described in U.S. Patent Application Ser. No. 60/987,913 as indicated previously and incorporated herein by reference.

The introducer 100 further comprises a cervical seal 120 located at a distal end thereof. In contrast to presently available cervical seals, the cervical seal 120 does not require selective actuation in order to engage with the walls of the cervical canal, as will be described in greater detail below. The cervical seal is formed as a flexible mechanical barrier extending from an outer diameter of an introducer 100 to seal a distal portion of the cervical canal 20 to prevent fluid transfer between the uterus and the proximal end of the cervical canal 20. As will be described in greater detail below, the cervical seal 120 comprises a plurality of flexible ribs 122 extending radially outward from the introducer 100 to enhance the engagement between the tissue of the cervical canal 20 and the device 100.

When the introducer 100 is in a desired position with a distal end 108 thereof within the uterus 14, the cervical seal 120 is seated within a distal portion of the cervical canal 20. The introducer 100 comprises a proximal elongated body 106 with a tapered portion 104 at a distal end thereof leading to a sheath 102 adapted to be received within the cervical canal 20. As indicated above, when in an operative position, a distal end 108 of the sheath 102 opens into the uterus 14. An outer diameter of the sheath 102 is preferably smaller than an outer diameter of the elongated body 106 and, in a preferred embodiment, is approximately 8 to 8.5 mm. The sheath 102 is formed of a polycarbonate material sufficiently rigid to allow the introducer 100 to be inserted through the cervical canal 20 into the uterus 14 as would be understood by those skilled in the art. Diameters of the delivery lumen 126 and the return lumen 128 extending through the sheath 102 may be maximized to or decreased to affect a flow of heated ablation fluid therethrough. The delivery lumen 126 and the return lumen 128 extend through the length of the introducer 100 and are open at a proximal portion comprising a handle 130 to a delivery inlet 134 and a return outlet 132 and, at a distal end, into the uterus. In use, a heated fluid source is connected to the delivery inlet 134 to pump fluid into a directed body cavity via the delivery lumen 126. After a predetermined duration (e.g., approximately 10 minutes), the heated fluid is removed from the body cavity via the return lumen 128 and out through the return outlet 132, which may be connected to a reservoir for receiving the heated ablation fluid.

The cervical seal 120 is separated from a distal end 108 of the sheath 102 by a length L₃ indicative of a preferred depth by which the introducer 100 is to abut into the uterus. The length L₃ was chosen to be approximately 5 mm to provide an unobstructed and clear translucent space through the base (tube) to cap for the UV bonding process. However, smaller lengths may be used. L₃ may be shortened for other processes where UV bonding is not used. The diameter at L₃ is a smaller (lead in diameter) to allow the tip of the introducer to be position into the canal before the slightly larger diameter of the seal is introduced. The distal end 108 is provided with a rounded edge which prevents trauma to a patient. The cervical seal 120 comprises a base 118 formed over a length of the sheath 102. The base 118 is formed of a silicone material with a thickness T, of approximately 0.1778 mm-0.2032 mm and an outer radius R, of approximately 3.886 mm. The base 118 may be formed of silicone or of the same material as the ribs 122 or, as would be understood by those of skill in the art, of any materials having sufficient flexibility at the ablation temperature and which is suitable for sterilization. Silicone rubber or a rubber like material may be desired for their properties such as silicone's resilience to our operating temperature of 90 degrees C. and its ability to recover to its normal (molded) state. Alternatively, acrylonitrile butadiene styrene (“ABS”), polyether block amide (“PEBA”) may be used as the material for the polycarbonate tube and the base mentioned below in this application. The material needs to be clear if UV bonding is to be used. In a preferred embodiment, an inner diameter of the base 118 and an outer diameter of the polycarbonate sheath 102, which contact one another in a bonded configuration as shown clearly in FIG. 3, can be plasma treated for approximately one minute, as those skilled in the art will understand, to form a bond therebetween. As would be understood by those skilled in the art, an open air ionized gas plasma treatment may be used. The activation energy for the plasma treatment may comprise long or short radio waves or may comprise microwaves, as also known to those skilled in the art. The plasma treatment prepares the contacting surfaces of the sheath 102 and the base 118 for bonding by raising the surface energy of each of the contacting surfaces, thus ensuring a sufficient bond therebetween. Once plasma treatment is complete, a small amount of isopropyl alcohol (“IPA”) is applied to the base 118 to allow the base 118 to slide over the plasma treated portion of the sheath 102. The sheath 102 may include markings aiding in positioning the base 118 thereon or the base 118 may be located on the sheath 102 by fixtures (e.g., by placement adjacent to a molded end connector of the sheath 102 as would be understood by those skilled in the art. An adhesive known in the art is then applied to the contacting surfaces of cuffs 124 located on ends of the base 118. The adhesive, such as, for example, Dymax 1-20779, is reactive to ultraviolet light and, once the base 118 of the cervical seal 120 has been properly positioned, is cured for approximately thirty seconds at 1836 mW/cm² power. Also a chamfer or radius is formed at the edge of a length L₁ of approximately 5.08 mm, as shown in FIG. 5. The transition radius which may, for example, be formed as a feathered out bead of bonding glue outside the end of the cuffs of the seal 118 provides a smoother/radius transition from the polycarbonate tube onto the silicone base 118. Alternatively, the base 118 can be secured to the sheath 102 via another means known in the art such as mechanical means (e.g., swaging, tying and sleeving, etc.). The base 118 is preformed with a plurality of circumferential ribs 122 located at equidistant intervals thereover. It is noted however, that any spacing interval may be provided between adjacent ribs 122. It is preferred that the spacing between the ribs 122 is large enough that adjacent ribs 122 do not overlap one another during introduction to minimize the collapsed profile. The ribs 122, which are formed of the same substantially flexible material as the base 118, comprise a thickness T₂ of approximately 0.2032 mm Each rib extends radially distally of the sheath 102 by approximately to an outer radius R₂ of approximately 5.499 mm, greater than the ribs free radius R₃ of approximately 5.296 mm. The embodiments of FIGS. 2-5 depict six ribs 122 formed on the base 118, each of the ribs 122 spaced from an adjacent rib 122 by a length L₂ of approximately 2.667 mm. It is noted, however, that any plurality of ribs 122 may be employed without deviating from the spirit and scope of the present invention. In one exemplary embodiment 16 ribs 122 may be employed to allow the entire canal to be in contact with the seal. The 16 ribs 122 may be formed longer than the canal to provide a seal during the hysteroscopy phrase where the sheath is moved and manipulated for viewing and to accommodate inadvertent movement during the ablation phrase. Using 16 ribs 122 provides a longer seal allowing the device to be positioned deep within the uterus, at user discretion, while maintaining the desired seal. The ribs 122, which are formed as a unitary element with the base 118, are permitted to deflect proximally or distally with respect to a longitudinal axis extending through the sheath 102. In this manner, the cervical seal 102 is movable between an initial diameter of approximately 11 mm and a compressed diameter of approximately 8.42 mm. when a pressure is applied to the ribs 122 by the wall of the cervical canal. The compressed configuration enables the cervical seal 102 to frictionally engage the wall of the cervical canal, which, as noted above, is dilated to a nominal 8 mm. Of course, those skilled in the art will understand that some cervical canals will exceed 8 mm in diameter even without dilation. The above embodiments of the seal may be overmolded onto the base 118 to avoid the gluing process.

In an alternate embodiment of the present invention, as shown in the partial cross-sectional view of FIG. 7, a cervical seal 220 is formed with substantially the same features of the cervical seal 120 of FIGS. 2-6. The cervical seal 220, which is formed as a cylindrical element is provided with a plurality of ribs 222 disposed along a plurality of lengths thereof. The ribs 222 are formed as rings encircling a base 218 of the cervical seal 220. Ends 224 of the ribs 222 are formed as protrusions along an inner diameter of the ribs 222. In the exemplary embodiment shown, the ends 224 are formed as triangular protrusions comprising a width greater than the width of the ribs 222 and housed in respectively shaped grooves 216 formed in the base 218. However, it is respectfully submitted that any suitable shape may be employed for the ends 224 without deviating from the spirit and scope of the present invention. For example, the ends 224 may be formed as spheres, rectangular prisms, etc. where the width of the end is at least greater than that of the ribs 222. By molding each of the ribs 222 into the base 218, the rigidity of the cervical seal 220 is strengthened while also bypassing the need for any additional bonding between the base 118 and the ribs 222.

A sheath 202 is machined over the cervical seal 220 via a means known in the art. For example, an outer wall of the sheath 202 in this area may be made thicker to accommodate machining triangular anchor slots thereinto. The sheath 202 is molded with a substantial friction fit to prevent leakage of any fluid therepast while still preventing a substantial radial constriction of the cervical seal 220. The machining process bypasses the need for any additional bonding means between the base 118 and the cervical seal 220. The sheath 202, which is formed of the same material as the cervical seal 220 defines a dual-lumen 226 extending therethrough from a proximal end (not shown) which is open to a heated ablation fluid source to a distal end (not shown) which is open to a uterus in an operative configuration, as described above with respect to FIGS. 2-6. Specifically, the dual-lumen 226 may comprise both a delivery lumen and a return lumen extending therethrough. Heated ablation fluid flowing through the dual-lumen 226 is substantially undeterred by the cervical seal 220 abutting thereinto due to a relatively negligible thickness thereof as also disclosed with respect to FIGS. 2-6.

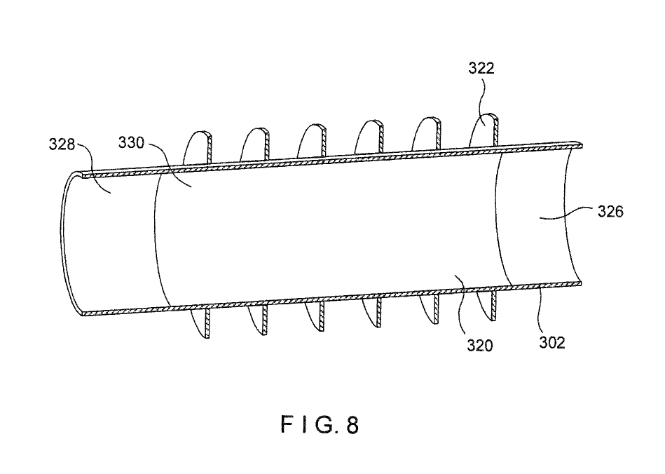

In a further embodiment, as shown in FIG. 8, a cervical seal 330 may be formed substantially similarly to the seal 220 of FIG. 5 except that the seal 330 is overmolded directly to the outer tube as described below to lie flush against an inner wall 328 of a dual-lumen 326 extending through a sheath 302. The sheath 302 is formed as an elongated hollow structure with a recess 316 formed along a length of a distal portion thereof, the recess 316 adapted to receive the cervical seal 320 therein. The cervical seal 320 is formed substantially similarly as shown in FIGS. 2-6, a base 318 thereof being formed as a unitary element with ribs 322. The sheath 302 is formed over the cervical seal 320 via an overmolding process known in the art. Once formed, an inner wall 330 of the cervical seal 320 lies flush against the inner wall 328 so that any fluid flow through the dual-lumen 326 is not obstructed.

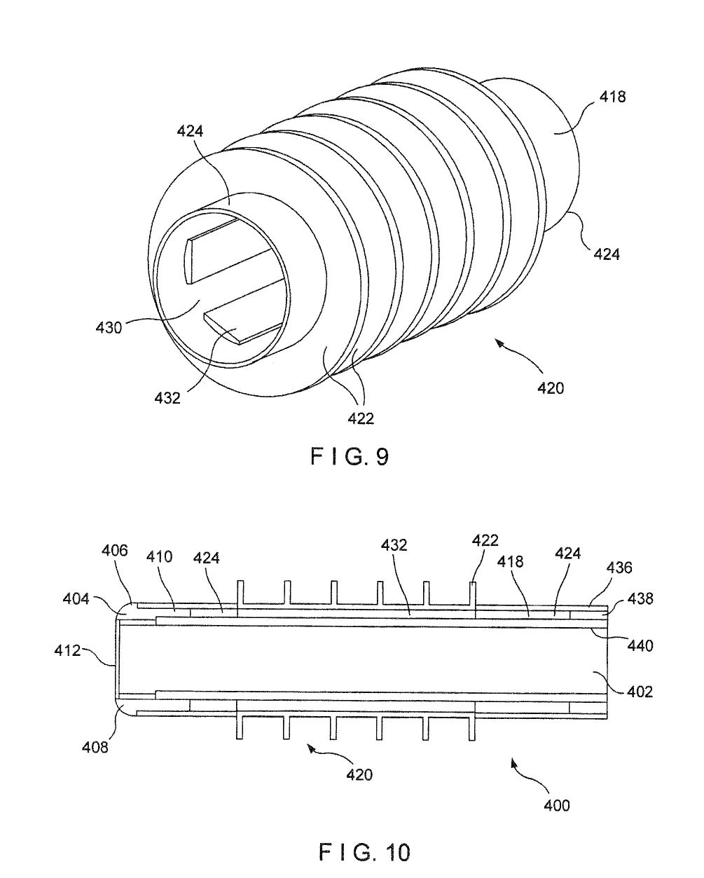

FIGS. 9-10 show yet another alternate embodiment of the present invention wherein a cervical seal 420 is formed by an overmolding process. Specifically, a plurality of ribs 422 are overmolded onto a base 418. An inner wall 430 of the cervical seal is provided with a plurality of longitudinal members 432 protruding into the base 418 running parallel to a longitudinal axis of the cervical seal 420. The base 418 has recesses in which the members 432 are formed with the members 432 locking the ribs 422 in place relative to the base 418 and is the connection between the ribs 422 during the molding process. Alternatively, the seal 420 may be overmolded on the base 418 obviating the need for the members 432. After the cervical seal 420 of FIG. 9 has been formed, a subsequent overmolding process is performed to join the cervical seal 420 with a sheath 402. The sheath 402, which may be formed with a smaller diameter than disclosed with respect to FIGS. 2-6, is received within the cervical seal 420 and bonded thereto. UV bonding is then performed to fit an outer sheath 436 over the subassembly, the outer sheath 436 defining a hollow portion 438 therein as well as a dual-lumen 440 extending longitudinally though a central axis thereof. Once the UV bonding process is complete, a distal end 408 of ablation device 400 is fitted with a stopper 404 with a substantial friction fit and UV bonded to seal the hollow portion 438 of the outer sheath 436. The stopper 404 is formed with a rounded distal end 406 in order to minimize any sharp edges on the ablation device which may cause trauma when inserted into a patient and a tapered leg 410 adapted to be received in the hollow portion 436. Once inserted, a proximal end of the tapered leg 410 engages a distal end of the base 418. The rounded edges of the stopper 404 converge at a central face 412 comprising a ring defining a distal opening of the ablation device. All the components in this embodiment may be designed to be inter-fitting and UV bonded to one another. UV bonding works best with transparent materials through which the UV light can pass. The seal 418 may then be over molded (e.g., in a single step). This design was to minimize the base of the seal to equal the diameter of the outer diameter of the sheath 436. The rigid surface of the base 418 allows it to be inserted into the ID of the sheath 436 for UV bonding.

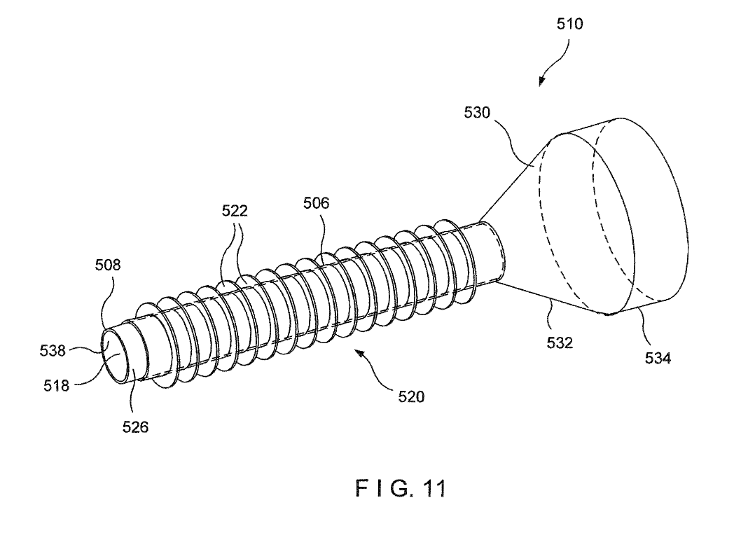

FIG. 11 depicts another embodiment of the present invention, comprising a cervical seal 520 formed on an accessory sheath 510 within which the known ablation device of FIG. 1 may be employed. Specifically, the accessory sheath of FIG. 11 may be received over a known ablation device adapted to be inserted into a cervical canal to perform a thermal ablation procedure. The accessory sheath 510 comprises the cervical seal 520 extending from an elongated body 506 fainted as a hollow tube. The elongated body 506 extends from a distal end 508 comprising an opening 518 to a proximal end comprising an increased diameter connector 530. The connector 530 follows a taper 532 increasing in diameter in a proximal direction away from the elongated body 506 to a substantially cylindrical proximal portion 534. An inner diameter of the proximal portion 534 is sized to be slightly greater than an outer diameter of an increased diameter portion of an ablation device such as, for example, the increased diameter portion 22 of the ablation device 10 of FIG. 1. In this manner, the connector 530 may be mated with the ablation device to form a secure fluid-tight seal.

A plurality of flexible ribs 522 are distributed along a portion of the length of the elongated body 506. The ribs 522 extend radially outward from the body 506 to engage tissue of a cervical canal, as described in greater detail earlier. Furthermore, a diameter and number of the ribs 522 may be adjusted to conform to the target portion of anatomy within which the device is to reside (e.g., the uterine cervix). Thus, dimensions of the device according to this embodiment may be similar to those disclosed above in regard to the cervical seal 120 of FIGS. 2-6. The ribs 522, elongated body 506 and the connector 530 which together comprise the accessory sheath 510, may be formed of a substantially flexible silicon material via an overmolding process, as those skilled in the art will understand. To provide the rigidity needed to slide the cervical seal 520 onto a thermal ablation device, the accessory sheath 510 may be overmolded onto a polyimide tube 526. In one exemplary embodiment, the polyimide tube comprises a diameter of approximately 0.0254 mm. An elongated introducer of a thermal ablation device such as, for example, the elongated tubular member 16 of ablation device 10, may then be inserted through the substantially rigid polyimide tube 526 and though a distal opening 538 thereof. A diameter of the polyimide tube 526 may be chosen to be slightly smaller than an outer diameter of the elongated tubular member 16 of the thermal ablation device 10 to ensure that a sufficient frictional seal is formed therebetween.

The device of FIG. 11 may be employed with a method substantially similar to the method discussed with respect to FIGS. 2-6. Additionally, since the accessory sheath 510 is selectively engageable with the thermal ablation device, a user of the device may decide when employment thereof becomes necessary. For example, the accessory sheath 510 may only be placed over the ablation device 10 when necessary (e.g., when a cervix is dilated to a diameter greater than a nominal 8 mm. diameter, if leakage occurs, etc.).

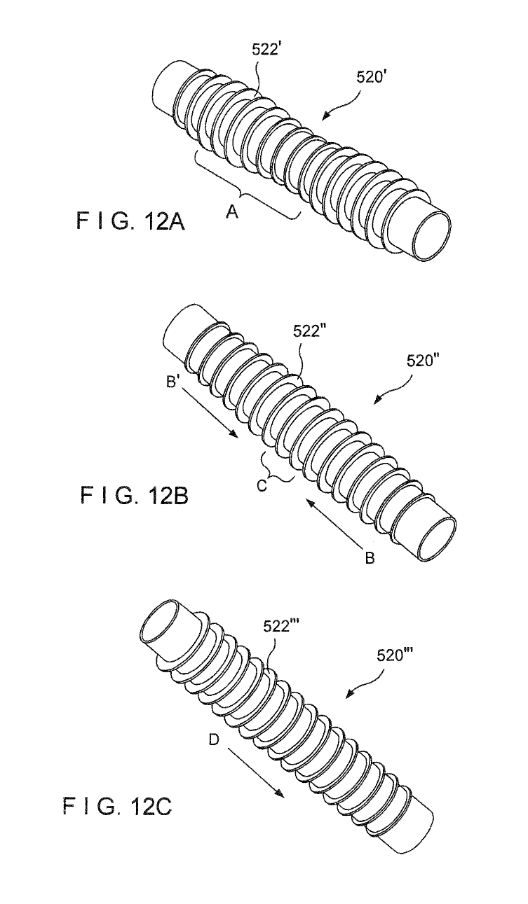

It is noted that although the accessory sheath 510 has been described with respect to thermal ablation procedure, the accessory sheath 510 and variations thereof may be employed in any other medical procedure requiring access to a uterus such as, for example, a biopsy, a tumor resection or a uterus flush, as those skilled in the art will understand. Furthermore, a plurality of modifications may be made to the cervical seal 520 without deviating from the spirit and scope of the present invention, As shown in FIGS. 12A-12C, ribs of the cervical seal 520 may assume any configuration and will not necessarily be circular. In one embodiment, as shown in FIG. 12A, the ribs 522′ of a cervical seal 520′ are formed in a dumbbell configuration with the ribs 522′ assuming a repeating pattern A and tapering down in size from a greater diameter to a smaller diameter. The ribs 522′ may fluctuate only from a predetermined maximum diameter to a predetermined minimum outer diameter. Employing the dumbbell shape of FIG. 12A may facilitate insertion of the cervical seal 520′ into a cervical canal while ensuring a sufficient frictional fit therewithin. In another embodiment, as shown in FIG. 12B, ribs 522″′ may comprise a minimum diameter at proximal and distal ends of the cervical seal 520″ tapering to a to a maximum outer diameter toward a center C of the cervical seal 520″. Specifically, the ribs 522′ located proximally of the center C may taper in the direction B and ribs 522′ located distally of the center C may taper in the direction B′. Furthermore, as shown in FIG. 12B, the ribs 522″ may be profiled into ovals or alternatively, any other shape to provide a better seal against surrounding tissue of the cervical canal. In another embodiment, as shown in FIG. 12C, ribs 522′ may taper down in a direction D from a first end of a cervical seal 520″′ to a second end.

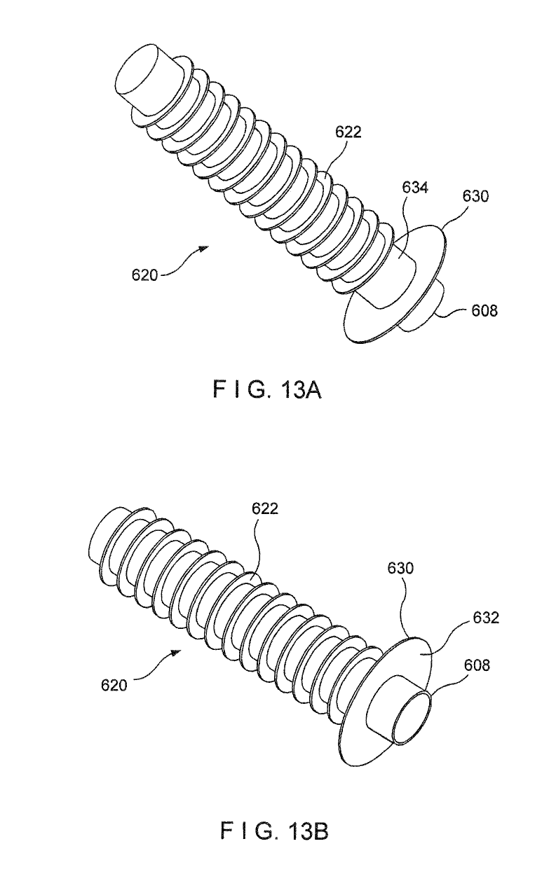

FIGS. 13A and 13B depict another alternate embodiment of the present invention wherein a distal portion of a cervical seal 620 is provided with a flap 630 located adjacent a distal end 608. The flap 630 is formed in substantially the same manner as ribs 622 and comprises substantially similar properties with the exception of a diameter thereof, which is selected to be greater than a diameter of an orifice of the cervical canal into the uterus, as those skilled in the art will understand. The flap 630 may be formed of the same substantially flexible silicon material as ribs 622. The flap 630 is introduced through the cervical canal and inserted into the uterus so that a proximal face 632 thereof abuts a wall of the uterus adjacent the orifice of the cervical canal. Thus, the flap 630 serves as an additional fluid sealing between the uterus and the cervical canal. Furthermore, a space 634 is provided between the flap 630 and a distalmost one of the ribs 622 to permit the flap 630 to deflect proximally during insertion through the cervical canal without overlapping over the distalmost one of the ribs 622. Those skilled in the art will understand that avoiding this overlapping prevents the profile of the cervical seal 620 from being unduly increased during insertion and reduces patient trauma.

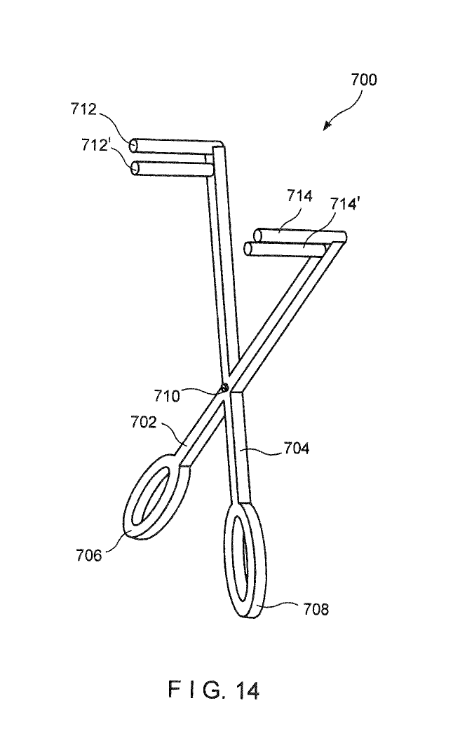

FIG. 14 depicts an exemplary expansion device 700 that may be used to place the accessory sheath 510 of FIG. 11 onto a thermal ablation device. The expansion device 700 is formed as a substantially scissor like device with two arms 702, 704 comprising finger-gripping means 706, 708 at proximal ends thereof. The arms 702, 704 are pivotally attached to one another by a hinge 710 separated from respective finger-gripping means 706 by a predetermined distance. A distal end of each of the arms 702, 704 comprises a pair of protrusions 712, 712′ and 714, 714′, respectively, extending substantially perpendicularly from the arms 702,704. The protrusions 712, 712′, 714, 714′ are formed with smooth outer walls and may comprise a substantially cylindrical shape. The protrusion 712 is spaced from the protrusion 712′ and the protrusion 714 is spaced from the protrusion 714′ other a portion of the length of the respective one of the 712, 714.

According to an exemplary method of the present invention, the protrusions 712, 712′, 714, 714′ of the expansion device 700 are inserted into an inner diameter of the accessory sheath 510. The arms 702, 704 are then manually actuated via the finger-gripping means 706, 708 to separate the respective distal ends from one another. The expansion causes the accessory sheath 510 to expand to a slightly greater diameter, thus allowing the elongated tubular member 16 of the thermal ablation device 10 to be inserted with minimal frictional resistance. Those skilled in the art will understand that the employment of multiple protrusions 712, 712′, 714, 714′ on the arms 702, 704 decreases a pressure applied to an adjacent portion of a wall of the accessory sheath 510. Specifically, the increase in abutting surface area between the protrusions and the wall of the accessory sheath 510 subjects a lesser expanding pressure on the wall of the accessory sheath 510. Once the accessory sheath 510 has been properly positioned over the elongated tubular member 16, the expansion device 700 is removed and the accessory sheath 510 recovers to its original diameter to assume a compressed configuration against the elongated tubular member 16.

It is noted that the embodiments shown are shown for illustrative and descriptive purposes only and are not intended to describe the bounds of the present invention which is to be limited only by the scope of the claims appended hereto. There are many modifications of the present invention which will be apparent to those skilled in the art without departing from the teaching of the present invention. For example, various modifications may be made to the material, size, shape and number of ribs employed in any of the embodiments discussed herein without deviating from the spirit and scope of the present invention.

1.-29. (canceled)

30. A thermal ablation device, comprising:
an elongated member sized and shaped for insertion into a uterus via a cervical opening, the elongated member defining a first lumen extending therethrough to a first distal opening which, when the apparatus is in an operative position, opens into the uterus and a second lumen extending therethrough to a second distal opening which, when the apparatus is in the operative position, opens into the uterus;
a base member positioned over a portion of the elongated member so that, when a distal end of the elongated member is open to the uterus, the base member is received within the cervical canal; and
a passive seal positioned on the base member, the seal including a plurality of deflectable flexible members separated from one another along a length of the base member, the deflectable members being permanently positioned to project radially outward from an outer surface of the base member so that, when the elongated member is in the operative position, the seal is adapted without actuation to engage cervical tissue around a circumference of the base member and prevent fluid introduced into the uterus via the first lumen from flowing therepast into the cervix.

31. The apparatus of claim 30, wherein the flexible members are substantially disc-shaped and are deflectable proximally and distally when compressed by contact with surrounding tissue.

32. The apparatus of claim 30, wherein the base member is an elongate cylindrical fitting bonded to the thermal ablation device.

33. The apparatus of claim 30, wherein the base member is an elongated cylindrical fitting molded into the thermal ablation device.

34. The apparatus of claim 30, wherein the seal is formed of silicone.

35. The apparatus of claim 30, where each of the flexible members is approximately 0.2 mm thick.

36. The apparatus of claim 30, wherein an outer diameter of each of the flexible members is approximately 11 mm.

37. The apparatus of claim 30, wherein when the second distal opening opens into the uterus, the second lumen is operative to withdraw fluid from the uterus.

38. The apparatus of claim 30, wherein each of the first and second lumens has a D-shaped cross-section.

39. A thermal ablation system, comprising:
an elongated member sized and shaped for insertion into a uterus via a cervical opening, the elongated member defining a first lumen extending therethrough to a first distal opening which, when the apparatus is in an operative position, opens into the uterus and a second lumen extending therethrough to a second distal opening which, when the apparatus is in the operative position, opens into the uterus, the first and second lumens having a D-shaped cross-section, the elongated member including a seal positioned on a base member provided on a distal portion of the elongated member, the seal including a plurality of deflectable members permanently positioned to project radially outward from an outer surface of the base member so that, when the elongated member is in the operative position, the seal is adapted without actuation to engage cervical tissue around a circumference of the base member and prevent fluid introduced into the uterus via the first lumen from flowing therepast into the cervix; and
a fluid handling unit fluidly coupled to a proximal end of the first lumen, the fluid handling unit supplying heated fluid to the first lumen.

40. The system of claim 39, further comprising an introducer coupled to a proximal end of the elongated member including a grip and a scope connector extending proximally therefrom.

41. The system of claim 40, wherein the grip is sized and shaped for single-handed use.

42. The system of claim 40, further comprising a tenaculum stabilizer located on a proximal portion of the introducer.

43. The system of claim 40, wherein a proximal end of the second lumen is in fluid communication with the fluid handling unit to withdraw fluid from the uterus.

44. The system of claim 43, wherein the fluid handling unit returns fluid withdrawn from the uterus to the first lumen for resupply to the uterus.

45. The system of claim 43, wherein the fluid handling unit forwards fluid from the second lumen to a waste collection unit.

46. The system of claim 39, wherein the seal is formed of silicone.

47. The system of claim 39, where each of the flexible members is approximately 0.2 mm thick.

48. The system of claim 47, wherein an outer diameter of each of the flexible members is approximately 11 mm.
